(19) United States
(12) Patent Application Publication
(10) Pub. No.: US 2016/0271045 A1
(43) Pub. Date: Sep. 22, 2016

(54) ANTI-PERSPIRANT COMPOSITION

(71) Applicant: CONOPCO, INC., D/B/A UNILEVER, Englewood Cliffs, NJ (US)

(72) Inventors: Mark John BERRY, Higham Ferrers (GB); John CASEY, Yelden (GB); Karl John HUNTER, Bedford (GB)

(73) Assignee: Conopco, Inc., d/b/a UNILEVER, Englewood Cliffs, NJ (US)

(21) Appl. No.: 15/030,386

(22) PCT Filed: Sep. 30, 2014
(86) PCT No.: PCT/EP2014/070911

§ 371 Date: Apr. 19, 2016

(30) Foreign Application Priority Data
Oct. 23, 2013 (EP) 13189911.4

(51) Int. Cl. A61K 8/73; A61K 8/26; A61Q 15/00

(52) U.S. Cl. CPC A61K 8/736; A61Q 15/00; A61K 8/26

(57) ABSTRACT

The invention relates to use of chitosan or a salt thereof in an anti-perspirant composition as an anti-perspirant ingredient. Current anti-perspirants are based on aluminium, but inorganic salts have the effect of leaving white patches on clothes. Additionally there is a perceived health risk associated with aluminium The current approach is to reduce the amount of aluminium in antiperspirants or to use additional metal salts such as those of zirconium. However, this approach tends to lower the efficacy of the formulation and hence prove more expensive. Zirconium-based antiperspirants tend to leave yellow patches on clothes. Use of chitosan or a salt thereof in an anti-perspirant composition as an anti-perspirant ingredient is provided, wherein the chitosan or salt thereof has a degree of acetylation of 0-15, preferably 0-12, most preferably 0-10%, wherein the chitosan or salt thereof is either in an anhydrous form or dissolved in water at a pH of no more than 6.0, preferably no more than 5.5, most preferably no more than 5.0.

The invention relates to use of chitosan or a salt thereof in an anti-perspirant composition as an anti-perspirant ingredient.

Current anti-perspirant ingredients are based on aluminium, but inorganic salts have the effect of leaving white patches on clothes. Additionally there is a perceived health risk associated with aluminium The current approach is to reduce the amount of aluminium in antiperspirants or to use additional metal salts such as those of zirconium. However, this approach tends to lower the efficacy of the formulation and hence prove more expensive. Zirconium-based antiperspirants tend to leave yellow patches on clothes.

US 2009/0016978 A1 (Courtois et al.) describes an antiperspirant composition comprising a carrier substance and a water-soluble or water-dispersible thiolated polymer. The prior art inventors believe that the thiol groups of the thiomer enable or enhance the polymer's ability to act as a mucoadhesive and that this ability enables or enhances the antiperspirant activity of the thiomer. “Mucoadhesives” are materials that can attach to mucin in a biological surface. The prior art inventors further believe that the antiperspirant activity results, at least in part, from the ability of the thiomers to act as pore blockers. The thiomers, when swollen by water, are thought to serve to as plugs that may, at least in part, block the exit of sweat from eccrine sweat glands. It is essential for the invention that the thiomer is water-soluble or water-dispersible in order for it to dissolve or disperse in eccrine sweat.

WO 03/042251 (The Procter & Gamble Company) discloses compositions comprising chitosan in the form of a network of nano-sized fibres. Traditional chitosan is usually semi-crystalline and only soluble in acidic medium, typically in a pH range of from 1 to 5 limiting homogeneous formulation. A process for producing the network of nano-sized fibres is described involving the steps of forming an aqueous solution, neutralising the chitosan just to the point of precipitation, and homogenising the resulting suspension. It was observed that the minimum concentration of chitosan to inhibit Malassezia furfur (yeast implicated in dandruff) was lower than expected. This document also discloses an anti-dandruff composition comprising from about 0.01% to about 5%, preferably from about 0.5% to about 2% of chitosan by weight of the composition as the active anti-dandruff agent. The chitosan can be used in different applications, such as hair care, skin care, personal cleansing, odour control, wound care, blood management, oral care, film formation, controlled release of hydrophobic or hydrophilic materials, hard surface, fabric treatment, plant care, seed, grain, fruit and food protection, water purification and drug delivery. The chitosan compositions provide hair care benefits when formulated into products such as shampoos, conditioners, hairsprays, styling mousses and gels, hair tonics and hair colorants, especially anti-dandruff benefits and reduction of hair damage caused by the process of hair bleaching, permanent waving or coloration. Additionally, the compositions provide scalp benefits and conditioning properties such as softening, manageability and stylising of the hair. Specific examples are a shampoo, a conditioner, a dentifrice, a mouthwash, a non-abrasive gel, a chewing gum and a plant care composition.

WO 2006/040092 (Beiersdorf AG) discloses an aerosol formulation comprising one or more anti-perspirants and/or deodorising substances and chitosan having a degree of deacetylation of 75 to 98%, a viscosity of 5 to 10 mPas, a weight average molecular weight distribution of less than 300 000 Da and a number average molecular weight distribution of less than 100 000 Da. It appears that the disclosed chitosan preserves the skin flora rather than acting purely as a bacteriocide. In particular, the chitosan appears to bind to the bacteria preventing microbial decomposition of sweat leading to odour. Anti-perspirants reduce sweat formation with the aid of astringent compounds in them, which are predominantly aluminium salts, such as aluminium hydrochloride, activated aluminium chlorohydrate or aluminium zirconium. It is customary to combine astringents with antimicrobials in the same composition. Aerosol products generally contain active anti-perspirant substances in the form of solids, which are suspended in an oil phase. Conventional active deodorant substances include ethyl hexyl glycerol, methyl phenyl butanol and polyglyceryl-2-caprate. One aim of the invention described in WO 2006/040092 is to reduce whiteness on skin or clothes. The formulation comprises 0.001-2, preferably 0.01-1, especially 0.015-0.3% w/w chitosan. The formulation comprises 1-35, preferably 1-25, especially 1-20% w/w anti-perspirant component. The formulation comprises preferably 0.01-10, especially 0.05-5% w/w deodorant component. Examples disclosed are anhydrous compositions. WO 2006/040092 further discloses that the pressure container used for the aerosol can be made of a metal, protected glass, non-shatter glass or some other glass, or else of a plastic. The propellant gas is preferably chosen from a long list of suitable gases.

US 2003/0133891 (Cognis Corporation) discloses a deodorising preparation containing nanoscale chitosans and/or chitosan derivatives with a particle diameter in the range from 10 to 300 nm. Chitosans have a bacteriostatic effect and a synergistic deodorising effect with esterase inhibitors and aluminium chlorohydrates. It is disclosed that absorption of nanoscale chitosans and/or chitosan derivatives by the Stratum Corneum is increased leading to long-lasting deodorising effect. The chitosan is normally used at levels of 0.01-5, preferably 0.1-1, more particularly 0.2-0.6% w/w. The document provides long lists of anti-perspirants based on salts of aluminium, zirconium or zinc, and deodorants. The preparations may contain 1-50, preferably 5-30, particularly 10-25% w/w anti-perspirants. Specific examples of anhydrous anti-perspirant or deodorant suspension sticks and soft solids, deodorant cream emulsions, and oil-in-water roll-on and sprayable anti-perspirants/deodorants are provided. In particular a composition (composition 2 in table 2) is disclosed comprising the nanoscale chitosan, distearyl ether and dioctyl carbonate.

WO 03/072610 (Cognis Deutschland GmbH & Co. KG) discloses transparent cosmetic preparations containing chitosan and having a pH of below 6, comprising a) chitosan and/or chitosan derivatives, b) at least one anionic surfactant, c) at least one alkyl oligoglycoside, and d) water. Chitosans are valuable raw materials for use in cosmetics, because they have film-forming and moisturizing properties. They are also known to inhibit the activity of esterase-producing bacteria, so they are often incorporated into deodorants as well. Previously, it had been difficult to use them simultaneously with anionic surfactants, owing to the positive charge on them, leading to precipitation, which made the resulting preparation turbid. The document provides lists of anti-perspirants and esterase inhibitors. The preparations may contain 1-50, preferably 5-30, particularly 10-25% w/w anti-perspirants. Transparent anti-perspirants are claimed in claim 9. Examples of water-based clear cosmetic preparations containing chitosan and anionic surfactants are provided.

U.S. Pat. No. 5,962,663 (Henkel KgA and Norwegian Institute of Fisheries and Aquaculture Ltd) discloses that known cationic biopolymers can be divided into two groups: the first group of products includes those which have a high degree of deacetylation, are soluble in organic acids and form low-viscosity solutions, but do not have satisfactory film-forming properties. The second group includes products which have a low degree of deacetylation, a relatively high molecular weight and good film-forming properties, but are poorly soluble in organic acids and, accordingly, are difficult to make up. The invention relates to new cationic biopolymers with an average molecular weight of 800,000 to 1,200,000 Da, a Brookfield viscosity (1 percent by weight in glycolic acid) below 5,000 mPas, a degree of deacetylation of 80 to 88 percent and an ash content of less than 0.3 percent by weight which are obtained by repeatedly subjecting crustacean shells to alternate acidic and alkaline degradation under defined conditions. Compared with known cationic biopolymers of the chitosan type, the new biopolymers form clear solutions and, at the same time, show excellent film-forming properties, despite their high molecular weight. The invention also relates to the use of the new biopolymers for the production of cosmetic and/or pharmaceutical formulations such as, for example, hair-care or skin-care preparations, hair-repair preparations and wound-healing preparations, in which they may be present in quantities of 0.01 to 5 percent by weight. Examples of water-containing skin care formulations consisting of a soft cream, moisturising emulsion, anti-wrinkle cream, restoration cream, intensive care, regeneration emulsion, intensive skin care fluid, high quality skin care fluid and skin tonic are provided.

U.S. Pat. No. 5,968,488 (Henkel KgA) discloses deodorizing preparations containing cationic biopolymers, aluminium chlorohydrate and esterase inhibitors. It has surprisingly been found that cationic biopolymers, preferably of the chitosan type, inhibit the activity of esterase-producing bacteria and that a synergistic deodorizing effect is obtained in conjunction with the two components mentioned above. The biopolymers have a bacteriostatic effect. At the same time, the use of the cationic biopolymers leads to an improvement in the dermatological compatibility of the products. Examples of water-based compositions are provided. U.S. Pat. No. 5,968,488 further discloses use of propellant gases for spray applications. The formulations are preferably marketed as rollers (roll-on emulsion), sticks, deodorant sprays or pump sprays.

FR 2 701 266 A (Jeon) discloses a biomedical grade of chitosan with a high degree of deacetylation and molecular weight. Examples 7 to 9 have a degree of deacetylation of ≧92% whilst Examples 10 to 12 have a degree of deacetylation of ≧85%.

EP 1 384 404 A (The Proctor & Gamble Company) discloses a hair-care composition comprising an anti-dandruff effective amount of anti-microbial oligoaminosaccharide comprising at least about 50 percent, preferably at least about 80 percent by weight of oligoaminosacharides having from 1 to 50 monomer units. The invention also relates to the use of the anti-microbial oligoaminosaccharide in a hair-care composition for providing anti-dandruff activity. The efficacy of aminosaccharides in oligomer form (i.e., less than 50 monomer units), especially chitosan oligomers, has been found to be superior to that of aminosaccharides in other forms, such as for example aminosaccharides in high molecular weight polymer form (i.e., more than 50 monomer units). The oligoaminosaccharides suitable for use herein are preferably soluble at ambient temperature (20 degrees centigrade) in aqueous solutions buffered (using for example acetate or one of the other primary pH standards of DIN 19266) to a pH from about 1 to about 10, preferably from 1 to 12. Preferred oligoaminosaccharides for use in the composition of the invention are selected from oligomers of chitosan (including isomeric modified forms), chitosan derivatives and mixtures thereof. A preferred chitosan oligomer for use herein is COS-Y LDA available from Primex. Chitosan oligomers not only present excellent anti-dandruff activity but also have a safe environmental profile. Low degree of acetylation is preferred for anti-dandruff efficacy. Chitosan oligomers for optimum anti-dandruff activity preferably have a degree of acetylation of less than about 30 percent. Example water-containing shampoos and hair conditioners are provided.

A number of products comprising, amongst other things, chitosan have been launched. Thus Laverana has launched a deodorant spray and roll-on under their Lavera brand in Germany. The product was also claimed as an anti-perspirant.

Jukona has launched a deodorant gel comprising, amongst other things, chitosan, under their Jukona Rose brand in Germany. It was claimed as free from aluminium salts.

Scholl has launched in Belgium an anti-perspirant foot spray comprising chitosan and aluminium chlorohydrate menthyl lactate.

Natura Cosmeticos has launched a roll-on anti-perspirant deodorant under their Natura Kaiak brand in Argentina comprising chitosan and aluminium chlorohydrate.

The inventors have observed that chitosan or a salt thereof is an effective anti-perspirant ingredient without the disadvantages of prior art anti-perspirant ingredients.

### SUMMARY OF THE INVENTION

Thus in a first aspect of the invention, use of chitosan or a salt thereof in an anti-perspirant composition as an anti-perspirant ingredient is provided, wherein the chitosan or salt thereof has a degree of acetylation of 0-15, preferably 0-12, most preferably 0-10%, wherein the chitosan or salt thereof is either in an anhydrous form or dissolved in water at a pH of no more than 6.0, preferably no more than 5.5, most preferably no more than 5.0.

For the purposes of this specification, the term “anti-perspirant composition” means a composition which prevents or reduces the appearance of perspiration or sweat in humans.

For the purposes of this specification, the term “anti-perspirant ingredient” means an ingredient which prevents or reduces the appearance of perspiration or sweat in humans.

For the purpose of this specification, the degree of acetylation is as measured using the dye-binding method (Gummow et al., Makromol. Chem., 186, 1239-1244 (1985)).

In a second aspect of the invention, a method of reducing or preventing perspiration is provided, the method comprising the step of topically applying an anti-perspirant composition comprising chitosan or a salt thereof as an anti-perspirant ingredient, wherein the chitosan or salt thereof has a degree of acetylation of 0-15, preferably 0-12, most preferably 0-10%, wherein the chitosan or salt thereof is either in an anhydrous form or dissolved in water at a pH of no more than 6.0, preferably no more than 5.5, most preferably no more than 5.0.

### BRIEF DESCRIPTION OF THE FIGURES

The invention is now described in more detail with reference to:

FIG. 1 which shows the effect of sweat pH on a variety of 0.1% w/v chitosan solutions (chloride counterion) all prepared at pH 5.

### DETAILED DESCRIPTION OF THE INVENTION

In a first aspect of the invention, use of chitosan or a salt thereof in an anti-perspirant composition as an anti-perspirant ingredient is provided, wherein the chitosan or salt thereof has a degree of acetylation of 0-15, preferably 0-12, most preferably 0-10%, wherein the chitosan or salt thereof is either in an anhydrous form or dissolved in water at a pH of no more than 6.0, preferably no more than 5.5, most preferably no more than 5.0.

Chitosan is a partially deacetylated form of the arthropod shell material chitin and is soluble in water at a pH of no more than 6.0. As well as from arthropods, chitosan and its precursor, chitin, are produced by fungi, thus potentially providing a non-animal source for chitosan from a by-product of the fermentation industry.

Without being bound by theory, it is thought that when chitosan or a salt thereof is applied to the skin, it can diffuse into pores where it comes into contact with sweat, which has a pH of approximately 7.7, and precipitates forming a gel blocking the pores and reducing sweat flow. The gel formed is not permanent as it is hydrolysed over time.

Preferred salts of chitosan are selected from the group consisting of acetate, chloride, citrate, formate, fumarate, gluconate, glycolate, lactate, maleate, malate, phosphate, propionate, succinate, sulphate, tartrate and mixtures thereof, preferably selected from the group consisting of formate, glycolate, lactate and mixtures thereof.

Preferably the anti-perspirant composition comprises 0.01-5, preferably 0.01-2, most preferably 0.01-1% w/w chitosan or chitosan salt.

The chitosan or salt thereof can be dissolved in water at a pH of at least 4.0, preferably 4.5.

In one embodiment the composition comprises chitosan, a salt thereof or a mixture thereof as the sole anti-perspirant ingredients.

Use according to any one of the preceding claims, wherein the composition additionally comprises auxiliary ingredients selected from the group consisting of a fragrance, a bactericidal agent, a bacteriostatic agent, a perspiration absorber, an esterase inhibitor, a surfactant, a thickener, a chelator and a preservative.

Suitable bactericides include chlorinated aromatics such as biguanide derivatives of which triclosan (e.g. Irgasan DP300 or Triclorban), and chlorhexidine warrant specific mention. Another class of effective bactericide comprises polyaminopropyl biguanide salts such as are available under the trade mark Cosmosil.

Chelators that can sequester iron retard bacterial growth and thereby inhibit malodour formation. Examples include aminopolycarboxylates such as ethylenediamine tetraacetic acid (EDTA) or higher homologues such as diethylenetriamine pentaacetic acid (DTPA).

Bactericides and chelators are commonly employed at a concentration of from 0.1 to 5, and particularly 0.1 to 2% w/w.

The composition can be in the form of a gel, or suitable for spray application, or suitable for application by aerosol, or suitable for application with a stick applicator. The method for their manufacture is well known to those skilled in the art.

In a second aspect of the invention, a method of reducing or preventing perspiration is provided, the method comprising the step of topically applying an anti-perspirant composition comprising chitosan or a salt thereof as an anti-perspirant ingredient, wherein the chitosan or salt thereof has a degree of acetylation of 0-15, preferably 0-12, most preferably 0-10%, wherein the chitosan or salt thereof is either in an anhydrous form or dissolved in water at a pH of no more than 6.0, preferably no more than 5.5, most preferably no more than 5.0.

The embodiments described hereinabove apply mutatis mutandis.

### EXAMPLES

### Example 1

### Break Pressure of Shrimp Chitosan

The break pressure, as a measure of the gel strength of shrimp chitosan in a pore, was measured compared to the performance from a conventional anti-perspirant agent aluminium chlorohydrate.

Materials:

Shrimp chitosan (Sigma-Aldrich C3646)

Aluminium chlorohydrate (ACH) (Sigma-Aldrich)

Method:

Chitosan chloride was prepared by adding the shrimp chitosan to water at 1% w/w to form a suspension. Hydrochloric acid was then added with stirring at room temperature until a stable pH of 5.0 was achieved. Undissolved chitosan was removed by centrifugation. The chitosan salt concentration was determined by precipitating chitosan using ammonium hydroxide. The resulting precipitate was then centrifuged at 13 000 g for 5 minutes at room temperature. The precipitate was then washed and centrifuged twice with 1 ml of 1 M ammonium hydroxide, and the precipitate dried under reduced pressure overnight. The resulting dry precipitate was then weighed to determine the initial concentration. The experimental concentrations were obtained by diluting the stock chitosan chloride with aqueous hydrochloric acid at a pH of 5.0 as necessary.

Artificial sweat was drawn into a glass capillary (10 μm diameter which is about the same as that of a human pore) under capillary action for one hour. The capillary was then placed in the test solution for one hour to allow diffusion into the capillary. It was then put onto the end of a vertical glass column, with the lower end of the capillary in a 20 ppm Phenol Red solution. Water was introduced into the top of the column until artificial sweat was seen leaking into the indicator, turning it from yellow to red. The height of the water was measured and converted into the pressure (mbar) needed to ‘break’ through the plug in the capillary aperture. The figures obtained were compared with aluminium chlorohydrate (a current anti-perspirant active) using the same protocol.

Artificial sweat was prepared as an aqueous solution (pH 7.7) consisting of:

160 mg·l⁻¹ Potassium chloride

1180 mg·l⁻¹ Sodium bicarbonate

840 mg·l⁻¹ Sodium chloride

212 mg·l⁻¹ Ammonium chloride

892 mg·l⁻¹ L-(+)-lactic acid

540 mg·l⁻¹ L-Methionine

52 mg·l⁻¹ Mucic acid

180 mg·l⁻¹ Urea

The pH of this solution (typically 6.0-6.2) was then adjusted to the desired pH by the drop wise addition of 0.01 M sodium hydroxide to raise the pH to 7.7.

Results:

The test solutions were aqueous solutions of chitosan chloride (pH 5.0) and aluminium chlorohydrate (pH unadjusted) both within a range of % w/w concentrations. The results are summarised in Table 1.

TABLE 1
Break pressures (mbar) for aqueous solutions of chitosan chloride
(pH 5.0) and aluminium chlorohydrate (pH unadjusted) at various
concentrations (n = 1) for 10 micron diameter capillaries.

| Test compound | % w/w | Break pressure (mbar) |
|---|---|---|
| Chitosan chloride | 0.000 | 11.8 |
| | 0.006 | 6.9 |
| | 0.018 | >37.2 |
| | 0.060 | >37.2 |
| | 0.180 | >37.2 |
| | 0.600 | >37.2 |
| Aluminium chlorohydrate | 0.000 | 7.8 |
| | 0.050 | 23.5 |
| | 0.200 | >37.2 |

37.2 mbar was the maximum pressure that could be applied using the vertical glass column. It was observed that as the concentration of chitosan increased, the break pressure increased reaching the maximum break pressure at a concentration in the range 0.006 to 0.018% w/w. The break pressure also increased as the concentration of aluminium chlorohydrate increased reaching the maximum break pressure at a concentration of 0.050 to 0.200% w/w.

Conclusion

An ex-vivo break pressure test has indicated that shrimp chitosan would be expected to be a better anti-perspirant active than conventional aluminium chlorohydrate at equal or lower molar and weight concentrations.

### Example 2

### Break Pressures of a Variety of Chitosans

The assessment described in Example 1 was expanded to include chitosans from other sources.

Materials (Additional):

Crab chitosan (Sigma-Aldrich 41865)

White mushroom chitosan (Sigma-Aldrich 740179)

White mushroom (Sigma Aldrich 740500)

Aspergillus niger (Clariant Zenvivo Aqua)

Aspergillus niger (Clariant Zenvivo Protect)

Method:

The method was as described for Example 1 except that the capillary was then placed in the test solution for two hours (rather than one hour) to allow diffusion into the capillary.

Chitosan chloride was prepared as previously described in Example 1. Chitosan acetate was prepared is similar fashion by substituting acetic acid for the hydrochloric acid used to prepare chitosan chloride.

Size exclusion chromatography was conducted by Reading Scientific Services Ltd. In brief, the method involved dissolving 20 mg of chitosan in 1% v/v aqueous formic acid. Polysaccharide reference standards were dissolved in the same diluent. Samples and standards were left to stand overnight to allow complete dissolution. Samples were prepared in duplicate. The analysis was carried out on an Agilent 1200 series HPLC equipped with an ELSD detector. The chromatographic separation was achieved on an Agilent PL aquagel-OH MIXED H, 300×7.5 mm ID, 8 μm particle size GPC column, using a buffer of 0.01 M aqueous ammonium formate (0.1% formic acid) at pH 3.1 as mobile phase, at a flow rate of 1.0 ml·min⁻¹.

The shear viscosities of the chitosans (with chloride counterion) were measured as 0.5% w/v aqueous solutions at a shear rate of 100 s⁻¹ using an Anton Paar MCR501 rheometer with a cone and plate configuration, a cone tip diameter of 50 mm and a gap distance of 0.049 mm.

Results:

Table 2 summarises the number average molecular weights and degree of acetylation of the test chitosans.

TABLE 2
Number average molecular weights and degree of acetylation
of test chitosans (n = 3 except white mushroom 740179)
(all sourced from Sigma-Aldrich).

| | | Viscosity of 0.5% w/v | |
|---|---|---|---|
| Chitosan source | | aqueous solution | Degree of |
| and Sigma-Aldrich | Molecular | (mPa · s at a shear | acetylation |
| code | weight (kDa) | rate of 100 s⁻¹) | (%)² |
| Crab, 41865 | 3,000¹/600³ | 141.4 | 0.0 ± 0.0 |
| Shrimp, C3646 | 1,400⁽¹⁾ | 29.54 | 10.1 ± 0.8 |
| White mushroom, | 140-220³ | 24.87 | 32.1 |
| 740179 | | | |
| White mushroom, | 110-150³ | 15.72 | 11.3 ± 6.7 |
| 740500 | | | |
| Aspergillus niger | 50-80³ | 5.02 | 1.1 ± 1.9 |
| (Clariant Zenvivo | | | |
| Aqua) | | | |
| Aspergillus niger | 10-20³ | 1.89 | 0.0 ± 0.0 |
| (Clariant Zenvivo | | | |
| Protect) | | | |

⁽¹⁾assayed using size exclusion chromatography;
²assayed by a dye-binding method (Gummow et al., Makromol. Chem., 186, 1239-1244 (1985)). Standard deviations are provided;
³company data.

Although the number average molecular weights of the four fungal chitosans have not been determined in-house, the degrees of acetylation of the two mushroom chitosans are significantly higher than those of the shrimp, crab and Aspergillus niger chitosans.

The test solutions were aqueous solutions of chitosan chloride (pH 5.0), chitosan acetate (pH 5.0) and aluminium chlorohydrate (pH unadjusted) all within a range of % w/w concentrations. The results are summarised in Table 3.

TABLE 3
Break pressures (mbar) for aqueous solutions of chitosan
chloride (pH 5.0), chitosan acetate (pH 5.0) and aluminium
chlorohydrate (pH unadjusted) at various concentrations for
10 micron diameter capillaries.

| Chitosan source and | | | Break pressure |
|---|---|---|---|
| Sigma-Aldrich code | Counter-ion | % w/w | (mbar) |
| Water control (pH 5.0) | | | 3.9 |
| | | | 8.8 |
| | | | 10.8 |
| | | | 18.6 |
| | | | 27.4 |
| Aluminium | Chlorohydrate | 0.050 | 23.5 |
| Crab, 41865 | Acetate | 0.005 | 26.5 |
| | | | >35.3 |
| | | | >41.2 |
| | | 0.010 | 6.9 |
| | | | >38.2 |
| | Chloride | 0.005 | 12.7 |
| | | | 12.7 |
| | | | 13.7 |
| | | 0.010 | 34.3 |
| | | | >41.2 |
| Shrimp, C3646 | Acetate | 0.005 | 13.7 |
| | | | 27.4 |
| | | | >41.2 |
| | | 0.010 | >38.2 |
| | | | >41.2 |
| | Chloride | 0.005 | 7.8 |
| | | | 13.7 |
| | | | >35.3 |
| | | 0.010 | 10.7 |
| | | | >38.2 |
| White mushroom, 740179 | Chloride | 0.005 | 10.8 |
| | | 0.010 | 6.9 |
| White mushroom, 740500 | Chloride | 0.005 | 16.7 |
| | | 0.010 | 14.7 |
| Aspergillus niger | Chloride | 0.0025 | >42 |
| (Clariant Zenvivo Aqua) | | 0.0050 | >42 |
| | | 0.0100 | >42 |
| Aspergillus niger | Chloride | 0.0025 | 26.5 |
| (Clariant Zenvivo Protect) | | 0.0050 | >42 |
| | | 0.0100 | >42 |

Although this method does produce a significant degree of variation within replicates, both crab and shrimp chitosans in the acetate salt form exhibited higher break pressures than aluminium chlorohydrate at concentrations of 0.005 and 0.010% w/w compared to aluminium chlorohydrate at a concentration of 0.05% w/w. However, both crab and shrimp chitosans in the chloride salt form exhibited higher break pressures than aluminium chlorohydrate only at a concentration of 0.010% w/w compared to aluminium chlorohydrate at a concentration of 0.05% w/w.

Aspergillus chitosans in the chloride salt form exhibited higher break pressures than aluminium chlorohydrate at concentrations of 0.0025, 0.005 and 0.010% w/w compared to aluminium chlorohydrate at a concentration of 0.05% w/w. However, white mushroom chitosans in the chloride salt form did not exhibit higher break pressures than aluminium chlorohydrate at concentrations of 0.005 and 0.010% w/w compared to aluminium chlorohydrate at a concentration of 0.05% w/w.

Conclusion

An ex-vivo break pressure test has indicated that the acetate and chloride salts of crustacean-derived (crab and shrimp) chitosans would be expected to be better anti-perspirant actives than conventional aluminium chlorohydrate at equal or lower molar and weight concentrations. Fungal chitosans in the chloride salt form would also be expected to be better anti-perspirant actives than aluminium chlorohydrate. In contrast the two white mushroom chitosans exhibited inferior performance than aluminium chlorohydrate in the break pressure test, albeit at lower concentrations, but did not improve when the concentrations were increased from 0.005 to 0.010% w/w. This could be due to the crustacean and Aspergillus chitosans having lower degrees of acetylation as indicated in table 2.

In particular, it appears that chitosan salts with a degree of acetylation in the range 0-10% as calculated using the dye-binding method described by Gummow et al. are better anti-perspirant actives than conventional aluminium chlorohydrate at equal or lower molar and weight concentrations.

### Example 3

### Study on Pore Blocking of Various Chitosans Using 141 Micron Capillaries

According to Wilke et al. (International Journal of Cosmetic Science, 29, 169-179 (2007)), the distribution of the sweat duct internal diameter varies from 10-120 μm, in order to test the effect of chitosans at the larger pore diameter size range, the break pressures of chitosans (as aqueous solutions at pH 5.0) in 141 micron diameter capillaries were determined.

Method:

This utilised 0.5 μl TLC dropper pipettes, manufactured by Camag and obtainable through VWR International, Lutterworth, UK. From the known volume (0.5 μl) and length of the capillary (3.2 cm) it was possible to calculate the internal diameter as 141 μm.

Artificial sweat, prepared according to Example 1, was drawn up the 141 μm capillary by capillary action and the capillary was noted to be full within 5 seconds. The capillary was then suspended in a solution of the active to be tested at the concentration and pH desired for a period of 1 hour. The capillary was then removed from the active solution and allowed to dry for approximately 15 minutes before the break pressure measurement was made. This permitted the observation of sweat breakthrough that would otherwise be masked by residual active solution on the outside of the capillary. The use of tissue to dry the capillary was avoided as this may have drawn out material from within the capillary.

The capillary to be measured for break pressure was inserted into a break pressure rig using the correct size adapter for the 141 μm capillary. The rig comprised a pressure sensor (OmegaDyne Inc., OH, USA, model PXM409, maximum of 178 mBar), with an instantaneous readout available on a computer screen using the software supplied by the sensor manufacturer (TRH Control, OmegaDyne Inc., OH, USA). The pressure at which a visual breakthrough of water from the tip of the capillary is achieved is noted. The hydrostatic pressure applied to the capillary was increased gradually at a rate of 0.05 ml/min until sweat was seen to emerge from the tip of the capillary. The pressure at which this occurred was noted and recorded.

Results:

The results are summarised in Table 4 and show that all the chitosans previously tested with 10 micron diameter capillaries in Example 2 showed some blocking of the 141 micron diameter capillaries.

Please note that the data does not satisfy the assumptions necessary for analysis techniques based on the Normal distribution to be valid. For example, the data are constrained by a maximum pressure value and is not free to vary past this. Thus analysis by mean and standard deviation is not justified. Instead used a standard non-parametric method (Wilcoxon Rank Sum test), which does not make Normality assumptions, has been applied to investigate the differences between break pressures.

TABLE 4
Break pressures (mbar) for 0.2% w/v aqueous solutions of
chitosan chloride except for crab which was 0.16% w/v
(maximum possible concentration) (pH 5.0) for 141 micron
diameter capillaries. Errors are standard error of the mean.

| Chitosan source and Sigma-Aldrich code | Break pressures (mbar) |
|---|---|
| Empty capillary (back pressure from | 6.3 ± 0.2 |
| capillary alone) | n = 5 |
| Water (pH 5) | 16.7 ± 1.0 |
| | n = 10 |
| Crab, 41865 | 111 ± 38 |
| | n = 3 |
| Shrimp, C3646 | >178 ± 0 |
| | n = 4 |
| White mushroom, 740179 | 61 ± 5.7 |
| | n = 4 |
| White mushroom, 740500 | 31 ± 1.8 |
| | n = 4 |
| Aspergillus niger (Clariant Zenvivo Aqua) | 28 ± 3.5 |
| | n = 3 |
| Aspergillus niger (Clariant Zenvivo Protect) | 26 ± 1.5 |
| | n = 3 |

Conclusion:

The ex-vivo break pressure test data at both 10 and 141 μm capillary diameters are considered relevant for the entire eccrine sweat duct and thus it appears that a variety of chitosans (chloride counterion) would be expected to function as anti-perspirant actives over the entire range of sweat duct sizes.

### Example 4

### The Effect of Chitosan Concentration on Pore Blocking Using 141 Micron Capillaries

The previously tested shrimp and mushroom (Sigma-Aldrich code 740179) chitosans as chlorides were dissolved in water at pH 5.0 at a range of concentrations.

Method:

The method of Example 3 was used.

Results:

The results are summarised in Table 5. The shrimp chitosan reaches a maximum pressure sensor reading at 0.2% w/v, whereas for the mushroom chitosan (Sigma-Aldrich code 740179), a value greater than 0.2% w/v is required. This reflects the differences in molecular weight and viscosity of the chitosans at the same concentration.

For crab chitosan, the maximum concentration achievable is 0.16% w/v and for this concentration a mean breakthrough value of 111±38 mBar was obtained. With the other chitosans (White Mushroom code 740500, Aspergillus Zenvivo Aqua and Zenvivo Protect), there were no significant differences in the breakthrough values with increases of concentration up to 0.93, 0.97 and 0.69% w/v, respectively. This implied that the lowest three molecular weight chitosans tested are less effective in blocking the wider 141 μm diameter capillaries as earlier data with 10 μm capillaries presented in Example 2 had shown a concentration effect with the two Aspergillus chitosans with the lowest molecular weight.

TABLE 5
Break pressures (mbar) for aqueous solutions of shrimp
and mushroom (Sigma-Aldrich code 740179) chitosan chlorides
at pH 5.0 at various concentrations for 141 micron diameter
capillaries. Errors are standard error of the mean.

| | Shrimp C3646 break | Mushroom 740179 break |
|---|---|---|
| Chitosan (chloride) | pressure (mbar) with | pressure (mbar) with |
| concentration (% w/v) | standard error of mean | standard error of mean |
| 0.0 | 18.4 ± 1.6 (n = 4) | 19.4 ± 1.5 (n = 2) |
| 0.005 | 22.3 ± 2.1 (n = 4) | 27.8 ± 1.6 (n = 3) |
| 0.02 | 30.1 ± 4.7 (n = 5) | 30.2 ± 4.1 (n = 3) |
| 0.05 | 44.9 ± 6.4 (n = 5) | 32.0 ± 3.5 (n = 3) |
| 0.10 | 89.6 ± 30.7 (n = 4) | 31.0 ± 0.2 (n = 3) |
| 0.20 | 178.2 ± 0.0 (n = 4) | 61.3 ± 5.7 (n = 4) |
| 0.30 | — | 153.0 ± 25.0 (n = 3) |
| 0.40 | — | 142.6 ± 35.4 (n = 2) |
| 0.50 | — | 178.0 ± 0.0 (n = 3) |
| 0.93 | — | 178.0 ± 0.0 (n = 3) |

Conclusion:

The effectiveness of pore blocking by chitosan is dependent on molecular weight with the higher molecular weight polymers being more effective.

### Example 5

### Solubility of Chitosan

The pH dependence of shrimp chitosan solubility was assessed and its pore blocking ability determined.

Method:

Shrimp chitosan (C3646) solutions at the required pH were obtained by dispersing shrimp chitosan (1% w/v) in 100 ml of deionised water and the resultant pH was measured as 9.6. The pH was lowered by addition of 0.1 M hydrochloric acid drop-wise until a stable pH reading of 6.2 was obtained for 5 minutes, at which point a 10 ml sample of the mixture was removed, centrifuged at 5200 g for 10 minutes and the supernatant collected. The remaining chitosan dispersion was then adjusted to pH 6.1 with further addition of 0.1 M hydrochloric acid, and when the pH was stable, the process of sampling and centrifugation was repeated to obtain a pH 6.1 sample. This process was repeated to obtain pH 6.0 and pH 5.9 samples.

The concentration of the chitosan solutions was determined by adding 1 ml to a weighed Eppendorf microfuge tube and adding 0.5 ml of 28% ammonium hydroxide. After mixing the tube and contents were centrifuged at 13,000 rpm for 5 minutes, after which the supernatant was removed and the pellet was washed twice in 1 M ammonium hydroxide with centrifugation at each step. After the second wash, the supernatant was removed and the pellet dried under vacuum overnight to remove residual ammonia/water. The tube containing the dried pellet was weighed and the concentration of chitosan determined.

The samples were also tested for their pore blocking effectiveness with the 141 micron diameter capillaries in accordance with the method described in Example 3.

Results:

The results are summarised in Table 6. It was observed that at pH 6.2, no chitosan was detected therefore it is assumed that this is above the pH at which shrimp chitosan is soluble. At pH 6.1 the maximum concentration of chitosan that was dispersed was 0.07% w/v whereas at 6.0 and 5.9 a level of 0.1% w/v was calculated.

The pore blocking ability of the samples at pH 6.0 and 6.1 seemed comparable, but that of the sample at pH 5.9 significantly better despite apparently comprising the same amount of shrimp chitosan as the sample at pH 6.0. This could imply that the gelation of chitosan by contact with a sweat of higher pH (pH 7.7) may be sensitive to the magnitude of the pH difference.

TABLE 6
Shrimp chitosan (chloride counterion) pH dependent
solubility and break pressure (mbar).

pH

| | 5.9 | 6.0 | 6.1 | 6.2 |
|---|---|---|---|---|
| Concentration | 0.1 | 0.1 | 0.07 | 0 |
| (% w/v) | | | | |
| Break pressure | 89.5 | 30.8 | 26.8 | 17.9 |
| (mbar) | | | | |
| Break pressure | 21.6 | 2.8 | 2.6 | 1.0 |
| standard error | | | | |
| of mean | | | | |
| Number of | 7 | 7 | 7 | 9 |
| break pressure | | | | |
| experiments | | | | |

Conclusion:

Shrimp chitosan is only partially soluble above pH 6.0 at a concentration of 0.1% w/v and hence has no pore blocking effect at this pH.

### Example 6

### Effect of Sweat pH

Human eccrine sweat pH is known to vary in the range 6.2 to 7.7 and the effect of this pH range on pore blocking was evaluated in 141 micron diameter capillaries.

Method:

The method used for the 141 micron diameter capillaries was that described in Example 3 except that the artificial sweat was prepared as described below.

The chitosans were those set forth in Table 2.

The artificial sweat was prepared in the manner described in Example 1 and the pH of this solution (typically 6.0-6.2) adjusted to the desired pH by the drop wise addition of 0.01 M sodium hydroxide to raise the pH to 6.7, 7.2, 7.7, >8, or 0.01 M hydrochloric acid to reduce the pH to <6.

Results:

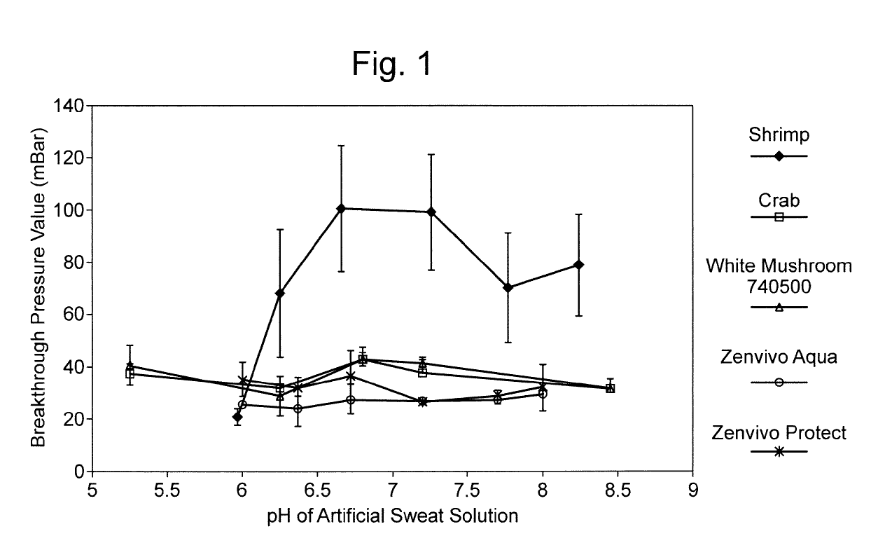

The results are summarised in FIG. 1.

For the shrimp chitosan, there was a significant increase in break pressure as the pH increased from 5.97 to 6.6. A decline in break pressure was then observed at pH 7.7, which could be due, according to Goycoolea et al. (Polymer Bulletin, 58, 225-234 (2007)), due to a change of state of the chitosan from a viscous solution to a crystalline solid at around pH 7.6.

For the lower molecular weight chitosans such as those from mushroom and Aspergillus niger, there is little pore blocking effect at the concentration of 0.1% w/v as used in this Example and thus, there is little variation in break pressure over the sweat pH range.

Conclusion:

The pore blocking effect of a range of chitosans is effective over the typical pH range of human eccrine sweat.

### Example 7

### Effect of Chitosan Counterion

The solubility of chitosan with a range of acids was evaluated and the effectiveness in pore blocking measured.

Method:

The solubility of 0.5% w/v shrimp chitosan C3646 in a range of 0.1 M acid solutions was assessed visually.

The pore blocking ability of shrimp chitosan C3646 with a variety of counterions was evaluated with 141 micron diameter capillaries. The concentrations of the chitosan varied from 0.05 to 0.20% w/v.

The method used for the 141 micron diameter capillaries was described in Example 3.

Results:

The shrimp chitosan C3646 was dissolved by acetic, fumaric, gluconic, glycolic, malic, maleic, propionic, succinic, formic, lactic and hydrochloric acids. The shrimp chitosan C3646 was mostly dissolved by phosphoric and tartaric acids. The shrimp chitosan C3646 was poorly dissolved by citric and sulphuric acids.

The results for break pressure are summarised in Table 7. The counterion appears to have little effect on the break pressure.

TABLE 7
Break pressure (mbar) for shrimp chitosan acidified with
various acids all at pH 5 with standard error of mean.
141 micron diameter capillaries.

| % w/v | Water | Chloride | Lactate | Formate | Glycolate | Acetate |
|---|---|---|---|---|---|---|
| 0.05 | — | 31.6 ± 2.8 | 55.9 ± 2.8 | 31.9 ± 3.9 | 30.9 ± 2.3 | 32.1 ± 1.6 |
| | | n = 4 | n = 5 | n = 5 | n = 6 | n = 4 |
| 0.10 | — | 74.5 ± 23.0 | 56.7 ± 23.0 | 42.6 ± 17.0 | 49.5 ± 18.4 | 67.1 ± 5.8 |
| | | n = 8 | n = 5 | n = 9 | n = 8 | n = 3 |
| 0.20 | — | 77.0 ± 19.2 | 63.1 ± 19.2 | 52.5 ± 4.7 | 83.6 ± 21.9 | 80.1 ± 49.1 |
| | | n = 10 | n = 7 | n = 9 | n = 8 | n = 3 |

↑ approximate value from extrapolation as upper limit of sensor exceeded.

Conclusion:

The nature of the counterion appears to have little effect on the break pressure.

### Example 8

### Effect of Aluminium Active

The effect of three commercial aluminium based antiperspirant actives was evaluated using the 141 micron diameter capillaries.

Materials:

Aluminium chloride (AlCl₃)

Activated Zirconium aluminium glycine (AZAG)

Method:

The method used for the 141 micron diameter capillaries was described in Example 3.

Results:

The results are summarised in Table 8.

For the 141 micron diameter capillaries, no real pore blocking effect was observed with any of the aluminium based actives. Whilst concentrations up to 20% w/v were also evaluated, pore blocking was still not improved. However the pH of these solutions was as low as pH 1 and therefore unlikely to have been gelled by a weak buffer artificial sweat.

TABLE 8
Break pressure (mbar) for AlCl₃, ACH and AZAG
standard error of mean. 141 micron diameter capillaries.

| | Water | % w/v | AlCl₃ | ACH | AZAG |
|---|---|---|---|---|---|
| | 18.7 ± 1.0 | 0.05 | — | — | 16.1 ± 1.4 |
| | n = 4 | | | | n = 6 |
| | — | 0.10 | 25.9 ± 0.6 | 17.5 ± 0.8 | 26.3 ± 3.1 |
| | | | n = 3 | n = 3 | n = 9 |
| | — | 0.20 | 26.9 ± 1.9 | 21.5 ± 2.0 | 32.7 ± 1.3 |
| | | | n = 4 | n = 3 | n = 2 |
| | — | 0.50 | 25.8 ± 0.9 | 26.0 ± 3.2 | 29.3 ± 0.9 |
| | | | n = 3 | n = 3 | n = 3 |
| | — | 1.00 | 26.5 ± 0.5 | 25.2 ± 0.6 | 31.5 ± 2.6 |
| | | | n = 2 | n = 2 | n = 3 |
| pH | 5.0 | | 4.6 | 5.0 | 5.0 |

↑ approximate value from extrapolation as upper limit of sensor exceeded.

Conclusion:

Three aluminium based actives, AlCl₃, ACH and AZAG, showed some pore blocking ability with 141 micron diameter capillaries.

### Example 9

### White Staining Evaluation

The white staining ability of shrimp chitosan compared to aluminium chlorohydrate was assessed on black cloth. For an accurate comparison for the staining of clothes in the underarm area, the form of the material after reaction with sweat was used, i.e. the aluminium hydroxide formed from aluminium chlorohydrate, and native chitosan formed from a chitosan salt (e.g. the chloride or acetate).

Materials:

Black cotton cloth

Method:

An aqueous solution of aluminium chloride was prepared by dissolving 10 mg of aluminium chloride in 100 ml of water. Aluminium hydroxide (formed when aluminium chlorohydrate reacts with sweat) was prepared as a gel by the addition of 10 ml of a 0.1 M aqueous sodium hydroxide solution to the solution of aluminium chloride. The resultant gel was washed with two aliquots of 100 ml of water to remove sodium salts, and two aliquots of 100 ml ethanol to remove residual water, the water and ethanol being separated by centrifugation. The resulting material was then re-suspended in 100 ml ethanol, and then dried to determine the % w/w concentration.

10 mg shrimp chitosan (Sigma-Aldrich, C3646) was suspended in 1 ml ethanol in a 2 ml microfuge tube containing glass beads (0.425-0.600 mm diameter) and whirly-mixed for 30 minutes to produce a fine suspension. After allowing the balls and larger fractions of chitosan to settle, the supernatant was removed to another tube. An aliquot of the supernatant was dried into a weighed tube to determine the % w/w concentration as 0.7 mg·ml⁻¹.

The aluminium hydroxide suspension was diluted in ethanol to 0.64 mg·ml⁻¹, which was equivalent in terms of aluminium content to 0.7 mg·ml⁻¹ aluminium chlorohydrate). 14 aliquots of 0.1 ml of the diluted aluminium hydroxide suspension or the shrimp chitosan supernatant were dripped onto a black cotton cloth, with hot air from a hair dryer being used to evaporate the ethanol between additions. This procedure gave the equivalent of 1 mg of aluminium hydroxide or shrimp chitosan deposited onto small and equal areas of the cloth.

L*a*b* (CIELAB) values of the cloth before and after application of the aluminium hydroxide or shrimp chitosan were obtained using a Konica Minolta Spectrophotometer CM-2600d.

Results:

The change in L*a*b* (CIELAB) values of the cloth before and after application of the aluminium hydroxide or shrimp chitosan are presented in table 4.

TABLE 4
Change in L*a*b* (CIELAB) values of the cloth before
and after application of equal weight amounts of aluminium
chlorohydrate (as aluminium hydroxide) or shrimp
chitosan onto a black cloth.

| Active | ΔL* | Δa* | Δb* |
|---|---|---|---|
| Aluminium hydroxide | 65.86 | −0.51 | −1.63 |
| Shrimp chitosan | 15.92 | −0.32 | −1.01 |

At equal levels of the active component, the aluminium salt stain was more than 4-fold ‘whiter’ than the shrimp chitosan stain, on the basis of ΔL* values.

Conclusion:

On the basis of the results, it would be expected that a shrimp chitosan containing antiperspirant would cause significantly less staining of clothing than an aluminium salt containing antiperspirant, even when shrimp chitosan was added at the same percentage weight as the aluminium salt.

1. Use of chitosan or a salt thereof in an anti-perspirant composition as an anti-perspirant ingredient, wherein the chitosan or salt thereof has a degree of acetylation of 0-40, preferably 0-15, more preferably 0-12, most preferably 0-10%, wherein the chitosan or salt thereof is either in an anhydrous form or dissolved in water at a pH of no more than 6.0, preferably no more than 5.5, most preferably no more than 5.0.

2. Use according to claim 1, wherein the salt of chitosan is selected from the group consisting of acetate, chloride, citrate, formate, fumarate, gluconate, glycolate, lactate, maleate, malate, phosphate, propionate, succinate, sulphate, tartrate and mixtures thereof, preferably selected from the group consisting of formate, glycolate, lactate and mixtures thereof.

3. Use according to claim 1 or claim 2, wherein the anti-perspirant composition comprises 0.01-5, preferably 0.01-2, most preferably 0.01-1% w/w chitosan or chitosan salt.

4. Use according to any one of the preceding claims, wherein the chitosan or salt thereof is dissolved in water at a pH of at least 4.0, preferably 4.5.

5. Use according to any one of the preceding claims, wherein the composition comprises chitosan, a salt thereof or a mixture thereof as the sole anti-perspirant ingredients.

6. Use according to any one of the preceding claims, wherein the composition additionally comprises auxiliary ingredients selected from the group consisting of a fragrance, a bactericidal agent, a bacteriostatic agent, a perspiration absorber, an esterase inhibitor, a surfactant, a thickener, a chelator and a preservative.

7. Use according to any one of the preceding claims, wherein the composition is in the form of a gel, or suitable for spray application, or suitable for application by aerosol, or suitable for application with a stick applicator.

8. A method of reducing or preventing perspiration, the method comprising the step of topically applying an anti-perspirant composition comprising chitosan or a salt thereof as an anti-perspirant ingredient, wherein the chitosan or salt thereof has a degree of acetylation of 0-15, preferably 0-12, most preferably 0-10%, wherein the chitosan or salt thereof is either in an anhydrous form or dissolved in water at a pH of no more than 6.0, preferably no more than 5.5, most preferably no more than 5.0.

9. A topical anti-perspirant composition for use in reducing or preventing perspiration, the topical anti-perspirant composition comprising chitosan or a salt thereof as an anti-perspirant ingredient, wherein the chitosan or salt thereof has a degree of acetylation of 0-40, preferably 0-15, more preferably 0-12, most preferably 0-10%, wherein the chitosan or salt thereof is either in an anhydrous form or dissolved in water at a pH of no more than 6.0, preferably no more than 5.5, most preferably no more than 5.0.
